(19) United States
(12) Patent Application Publication
(10) Pub. No.: US 2025/0130211 A1
(43) Pub. Date: Apr. 24, 2025

(54) METHOD OF SCREENING NEW PSYCHOACTIVE SUBSTANCE AND PLATFORM THEREOF

(71) Applicant: National Taiwan University, Taipei (TW)

(72) Inventors: Cheng-Chih Hsu, Taipei (TW); Wei-Hsin Hsu, Taipei (TW); Kai-Wen Cheng, Taipei (TW); Hsin-Bai Zou, Taipei (TW); Tzu-Hsuan Feng, Taipei (TW)

(73) Assignee: National Taiwan University, Taipei (TW)

(21) Appl. No.: 18/436,249

(22) Filed: Feb. 8, 2024

(30) Foreign Application Priority Data
Oct. 23, 2023 (TW) 112140465

(51) Int. Cl. G01N 30/92; G01N 30/86; H01J 49/04; H01J 49/42

(52) U.S. Cl. CPC G01N 30/92; G01N 30/8679; G01N 30/8693; H01J 49/0459; H01J 49/4225

(57) ABSTRACT

A method of screening new psychoactive substance is provided, including providing a sample; placing the sample on chromatographic paper; ionizing the sample on the chromatographic paper by a direct analysis in real time (DART); performing a mass spectrometry analysis on the ionized sample to obtain a sample mass spectrum; and comparing a known standard mass spectrum with the sample mass spectrum, in which when a profile of the known standard mass spectrum is the same as a profile of the sample mass spectrum and the known standard mass spectrum is not exactly the same as the sample mass spectrum, the sample is determined to be the new psychoactive substance. A platform for screening new psychoactive substance is also provided to quickly screen out the new psychoactive substance.

### CROSS-REFERENCE TO RELATED APPLICATION

This application claims priority to Taiwan Application Serial Number 112140465, filed on Oct. 23, 2023, the disclosures of which are incorporated herein by reference in their entireties.

### BACKGROUND

### Field of Invention

The present disclosure relates to a method and a platform. More particularly, the present disclosure relates to a method of screening new psychoactive substance and platform thereof.

### Description of Related Art

Drug abuse is a severe social problem worldwide. Particularly, the issue of new psychoactive substances (NPS) increasingly emerged. NPS are structural or functional analogs of traditional illicit drugs, such as cocaine, cannabis, and amphetamine; these molecules provide the same or more severe neurological effects. Usually, immunoassays are utilized in the preliminary screening method. However, NPS obtain poor detectability to commercially available immunoassay kits.

Currently, the detection methods for NPS mainly use liquid chromatography tandem mass spectrometry (LC-MS/MS) for analysis. However, sample analysis and chromatography take a long time, and a large amount of organic solvents will be used.

Therefore, how to establish a rapid and objective platform for NPS screening, and the related art really needs to be improved.

### SUMMARY

The present disclosure provides a method of screening new psychoactive substance, comprising: providing a sample; placing the sample on a chromatographic paper; ionizing the sample on the chromatographic paper by a direct analysis in real time (DART); performing a mass spectrometry analysis on the ionized sample to obtain a sample mass spectrum; and comparing a known standard mass spectrum with the sample mass spectrum, wherein when a profile of the known standard mass spectrum is the same as a profile of the sample mass spectrum, and the known standard mass spectrum is not exactly the same as the sample mass spectrum, the sample is determined to be the new psychoactive substance.

In some embodiments, an ionization temperature of the direct analysis in real time is from 300° C. to 400° C.

In some embodiments, the direct analysis in real time comprises ionizing by a positive ionization mode.

In some embodiments, the direct analysis in real time comprises ionizing by a multiple reaction monitoring (MRM) mode in the positive ionization mode.

In some embodiments, a known standard of the known standard mass spectrum comprises a known drug.

In some embodiments, wherein the known drug comprises ketamine, methoxetamine, norketamine, deschloroketamine, mephedrone, 4-methylpentedrone (4-MPD), 4-methyl-α-ethylaminopentiophenone (MEAP), chloromethcathinone (CMC), 3,4-methylenedioxy-N-methylcathinone (methylone), ephylone, eutylone, 3,4-Methylenedioxy-α-pyrrolidinohexiophenone (3,4-MDPHP), amphetamine, methamphetamine, 6-acetylmorphine, 3,4-methylenedioxyamphetamine (MDA), 3,4-methylenedioxymethamphetamine (MDMA), para-methoxyethylamphetamine (PMEA), para-methoxyamphetamine (PMA), para-methoxymethamphetamine (PMMA), or a combination thereof.

In some embodiments, the mass spectrometry analysis is an ion trap mass spectrometry analysis.

In some embodiments, the profile of the known standard mass spectrum comprises one or more than one mass to charge ratio (m/z).

The present disclosure also provides a platform of screening new psychoactive substance, comprising a carrier, a DART device, a mass spectrometer, and a drug screening model. The carrier loads at least one chromatographic paper. The DART device is adjacent to the carrier. The mass spectrometer is adjacent to the DART device, in which a sample placed on the chromatography paper and ionized by the DART device is analyzed by the mass spectrometer to obtain a sample mass spectrum. The drug screening model comprises a computer processor and a memory, the memory stores a plurality of computer program instructions that, when executed by the computer processor, cause the computer processor to implement following steps, comprising: importing the sample mass spectrum; and comparing a known standard mass spectrum with the sample mass spectrum, wherein when a profile of the known standard mass spectrum is the same as a profile of the sample mass spectrum, and the known standard mass spectrum is not exactly the same as the sample mass spectrum, the sample is determined to be the new psychoactive substance.

In some embodiments, the at least one chromatographic paper is a plurality of chromatographic papers, wherein the carrier comprises a body and a plurality of paper modules sequentially disposed on the body, wherein the plurality of chromatography papers are respectively disposed on the plurality of paper modules.

In some embodiments, the mass spectrometer comprises a high-resolution mass spectrometer or a low-resolution mass spectrometer, in which the high-resolution mass spectrometer provides a mass accuracy level below 5 ppm and a mass resolution above 10,000 m/Δm; the low-resolution mass spectrometer provides a mass accuracy level above or equal to 5 ppm and a mass resolution below or equal to 10,000 m/Δm.

In some embodiments, the low-resolution mass spectrometer comprises a triple quadrupole linear ion trap mass spectrometer.

In some embodiments, the profile of the known standard mass spectrum comprises one or more than one mass to charge ratio (m/z).

In some embodiments, a known standard of the known standard mass spectrum comprises a known drug.

In some embodiments, the known drug comprises ketamine, methoxetamine, norketamine, deschloroketamine, mephedrone, 4-MPD, MEAP, CMC, methylone, ephylone, eutylone, 3,4-MDPHP, amphetamine, methamphetamine, 6-acetylmorphine, MDA, MDMA, PMEA, PMA, PMMA, or a combination thereof.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIG. 1 depicts a perspective view of a platform for screening new psychoactive substance according to some embodiment of the present disclosure.

FIG. 2 illustrates the relationship between total sampling volume and ketamine-d4 conversion AUC according to some embodiments of the present disclosure.

FIG. 3 illustrates the signal differentiation of different concentrations of mephedrone and the blank control group according to some embodiments of the present disclosure.

FIG. 4 is a scatter plot of comparison of concentrations for LC-MS/MS and pDART methods according to some embodiment of the present disclosure.

### DETAILED DESCRIPTION

The following disclosure provides detailed description of many different embodiments, or examples, for implementing different features of the provided subject matter. These are, of course, merely examples and are not intended to limit the disclosure but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

A number of examples are provided herein to elaborate the method of screening new psychoactive substance and platform thereof of the instant disclosure. However, the examples are for demonstration purpose alone, and the instant disclosure is not limited thereto.

Although a series of operations or steps are used below to describe the method disclosed herein, an order of these operations or steps should not be construed as a limitation to the present disclosure. For example, some operations or steps may be performed in a different order and/or other steps may be performed at the same time. In addition, all shown operations, steps and/or features are not required to be executed to implement an embodiment of the present disclosure. In addition, each operation or step described herein may include a plurality of sub-steps or actions.

### Method of Screening NPS

In some embodiments, the present disclosure provides a method of screening new psychoactive substance, including providing a sample. In some examples, the sample includes, bust is not limited to urine, blood, sputum, saliva, gastric juice or oral mucosa.

In some embodiments, the present disclosure provides a method of screening new psychoactive substance, including placing the sample on a chromatographic paper. As used herein, “chromatographic paper” is specially used in chromatographic technology and chromatographic paper enables the separation and analysis of components in a mixture based on their interactions with the stationary phase. On the other hand, filter paper is mainly used to physically separate solids from liquids or gases. Chromatography paper facilitates component separation while filter paper focuses on removing impurities from the sample.

In some embodiments, the present disclosure provides a method of screening new psychoactive substance, including ionizing the sample on the chromatographic paper by a direct analysis in real time (DART). In some examples, an ionization temperature of the direct analysis in real time is from 300° C. to 400° C. As used herein, “direct analysis in real time (DART)” is one of ambient ionization mass spectrometry (AIMS), that is a plasma-based ion generation technique that operates under a native environment. The ability of DART to efficiently ionize nonpolar small molecules allows it to readily in multiple forensic applications.

In some embodiments, the present disclosure provides a method of screening new psychoactive substance, including performing a mass spectrometry analysis on the ionized sample to obtain a sample mass spectrum. In some examples, the mass spectrometry analysis can analyze by a mass spectrometer, the mass spectrometer comprises a high-resolution mass spectrometer or a low-resolution mass spectrometer. In some examples, the mass spectrometry analysis is an ion trap mass spectrometry analysis.

As used herein, “low-resolution mass spectrometer (LRMS)” refers to a mass accuracy level above or equal to 5 ppm and a mass resolution below or equal to 10,000 FWHM. Mass accuracy is determined through ppm error: (measured mass−theoretical mass)/theoretical mass. The mass resolution is the capacity of a mass spectrometer to separate ions of close m/z ratios. It is defined as the ratio of the measured mass “m” to “Δm”, the full width of the peak at half its maximum height (i.e., m/Δm, FWHM). Mass analyzers of LRMS can typically be categorized as ion-trap (IT)-based or quadrupole (Q)-based mass analyzers. A combination of both analyzers (e.g. Q-IT) or several of the same mass analyser (e.g. QqQ) would also be possible. There are currently various forms of IT-based mass analyzers. It could be a linear, rectilinear or cylindrical ion trap. On the other hand, quadrupole-based mass analyzers are usually single or triple quad (QqQ). Else, they would be coupled with ITs such as quadrupole ion traps. Furthermore, as the field is continuously developing, these analyzers would one day able to achieve more than what it is capable of now.

As used herein, “high-resolution mass spectrometers (HRMS)” can be clearly defined as: mass spectrometers that are able to provide mass accuracy level below 5 ppm*, and a mass resolution above 10,000 (m/Δm, FWHM). The type of mass analyzer usually determines if the mass spectrometer is capable of acquiring high-resolution data. For instance, OrbiTrap, FT-ICR, and time-of-flight (TOF) instruments, in their optimum condition, will produce data that fulfils the criteria of HRMS.

In some embodiments, the present disclosure provides a method of screening new psychoactive substance, including comparing a known standard mass spectrum with the sample mass spectrum, in which when a profile of the known standard mass spectrum is the same as a profile of the sample mass spectrum, and the known standard mass spectrum is not exactly the same as the sample mass spectrum, the sample is determined to be the new psychoactive substance. As used herein, “not exactly the same” refers to that a specific one or more features in the known standard mass spectrum are the same as the corresponding one or more features in the sample mass spectrum, and the remaining features may be different. For example, a mass-to-charge ratio (m/z) in the known standard mass spectrum has a neuronal excitatory effect, and the sample mass spectrum also has a corresponding mass-to-charge ratio. Except for the same mass-to-charge ratio as above mentioned, the other mass-to-charge ratios can be partially the same or completely different. For another example, multiple mass-to-charge ratios (m/z) in the known standard mass spectrum have a neuronal excitatory effect, and the sample mass spectrum has one or more corresponding mass-to-charge ratios. Except for the same mass-to-charge ratio as above mentioned, other mass-to-charge ratios may be partially the same or completely different.

In some examples, the profile in the known standard mass spectrum includes one or more than one mass-to-charge ratio (m/z). The known standard of the known standard mass spectrum comprises a known drug. In some examples, the known drug comprises ketamine, methoxetamine, norketamine, deschloroketamine, mephedrone, 4-MPD, MEAP, CMC, methylone, ephylone, eutylone, 3, 4-MDPHP, amphetamine, methamphetamine, 6-acetylmorphine, MDA, MDMA, PMEA, PMA, PMMA, or a combination thereof.

### Platform of Screening NPS

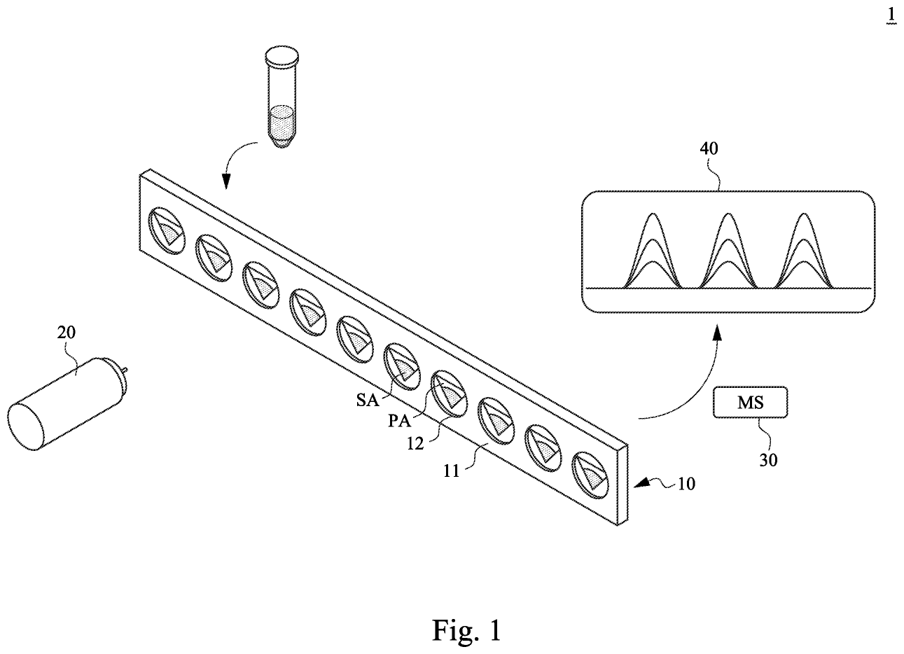

Please refer to FIG. 1, FIG. 1 depicts a perspective view of a platform for screening new psychoactive substance according to some embodiment of the present disclosure. The platform 1 of screening new psychoactive substance of the present disclosure includes a carrier 10, a DART device 20, a mass spectrometer 30, and a drug screening model 40.

The carrier 10 loads at least one chromatographic paper PA. In some examples, the at least one chromatographic paper PA is a plurality of chromatographic papers PA. In some examples, the carrier 10 comprises a body 11 and a plurality of paper modules 12 sequentially disposed on the body 11, in which the plurality of chromatography papers PA are respectively disposed on the plurality of paper modules 12. The carrier 10 has an axial translational motor that can make these paper modules 12 move alone the axial. In some examples, the carrier 10 includes, but is not limited to automatic transmission module (IonSense, Saugus, MA).

The DART device 20 is adjacent to the carrier 10. In some examples, a DART SVP ion source (IonSense, Saugus, MA) interfaced to the mass spectrometer 30 via a VAPUR interface (IonSense, Saugus, MA) was utilized.

The mass spectrometer 30 is adjacent to the carrier 10, a sample placed on the chromatography paper PA and ionized by the DART device 20 was analyzed by the mass spectrometer 30 to obtain a sample mass spectrum. In some examples, the mass spectrometer 30 includes, but is not limited to a HRMS or a LRMS. The HRMS provides a mass accuracy level below 5 ppm and a mass resolution above 10,000 m/Δm, and the LRMS provides a mass accuracy level above or equal to 5 ppm and a mass resolution below or equal to 10,000 m/Δm. In some examples, the LRMS includes a triple quadrupole linear ion trap mass spectrometer.

The drug screening model 40 includes a computer processor and a memory, the memory stores a plurality of computer program instructions that, when executed by the computer processor, cause the computer processor to implement following steps, comprising: importing the sample mass spectrum; and comparing a known standard mass spectrum with the sample mass spectrum, in which when a profile of the known standard mass spectrum is the same as a profile of the sample mass spectrum, and the known standard mass spectrum is not exactly the same as the sample mass spectrum, the sample is determined to be the new psychoactive substance.

### Biological Sample Collection

Blank urine samples were obtained from 7 normal healthy volunteers, 3 males, and 4 females. 40 urine samples were collected from intoxicated patients. Neat urine specimens were collected in sample collectors and were stored at −20° C. until screened. Except for vortexing, no further sample pretreatment was conducted on all specimens.

### Sample Preparation

All test solutions, including urine specimens and spiked standard solutions at the desired levels of analyzed compounds, were added with the mixtures of deuterated internal standards. The final concentration of the internal standard was kept constant in all the samples and blanks at 90.91 ng/mL of each standard (i.e. 100 μL of standard solution/human urine was collected and 10 μL of the standard solution was pipetted into the human urine sample, then mixed well). The chromatography paper was sliced into equilateral triangular pieces (side lengths of 1.0 cm) by paper punch, then was fixed on the paper module 12 of the carrier 10 of the platform 1; the working solution (such as the samples and blanks) was carefully loaded onto the apex of the chromatographic paper PA triangle and then left untouched until it was dried; until the paper-loaded samples were dried, the paper module 12 was installed to the axial translational body 11. The measurement was conducted at the rate of 0.4 mm/sec against ten samples per test set.

### DART-MS Analysis, and Data Processing

A DART SVP ion source (IonSense, Saugus, MA) interfaced to the mass spectrometer via a VAPUR interface (IonSense, Saugus, MA) was utilized throughout all the pDART-MS experiments in the present disclosure. Ultrahigh purity nitrogen (99.999%) was used as the standby gas, and ultrahigh purity helium (99.999%) served as the running gas. The output pressures of both nitrogen and helium were set at 0.5 MPa. Grid Voltage was 250V in positive mode. The gas heater temperature of the helium reagent gas was set dependent experiment. An automatic transmission module (IonSense, Saugus, MA) was used as a paper holder carrying 10 paper triangles sliced by a commercial paper cutter in one set of experiments.

All experiments were performed utilized LRMS: Applied Biosystems SCIEX Triple Quad 5500 System and the SCIEX QTRAP 5500 System (AB SCIEX 5000 Q TRAP). The mass spectral analysis and data were collected and processed with the software package of Analyst 1.6.3 (AB SCIEX). The quantitative validation of drugs of abuse and real sample analysis were analyzed by multiple reaction monitoring (MRM) mode in a positive ionization mode. All the parameters of analytes were collected under the flow injection analysis with electrospray ionization (ESI) resource. The MS inlet capillary temperature and voltage were kept at 300° C. and 35 V, respectively. Prior to application on paper-loaded DART analysis, isobaric interferences on chromatography paper were assessed to evaluate the selectivity of the transitions. Two to four transitions were selected for each compound, and the optimized declustering potential (DP), entrance potential (EP), collision energies (CE), collision cell exit potential (CXP) are summarized in Table 1. One quantifier transition was chosen based on the comprehensive validation result whereas the other transitions were used for the identification of the compounds. The peak area of the analyte fragment ion over the entire analysis time was determined by manual peak integration using Sciex OS (AB SCIEX).

TABLE 1
MRM transitions and experimental parameters for all analytes and internal standards

| Compound | | Precursor ion [M + H]⁺ | DP | EP | Fragment | m/z | CE | CXP | Internal standard | Normalization fragment | Ionization helium temperature |
|---|---|---|---|---|---|---|---|---|---|---|---|
| 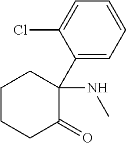 [SMILES: CNC1(c2ccccc2Cl)CCCCC1=O] | Ketamine | 238 | 80 | 10 | Quantifier | 125 | 40 | 10 | Ketamine-D4 | 242/129 | 300 |
| | | | | | Qualifier | 220 | 22 | 10 | | | |
| | | | | | Quantifier | 203 | 19 | 14 | | | |
| 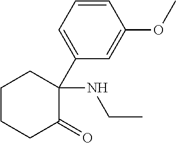 [SMILES: CCNC1(c2cccc(OC)c2)CCCCC1=O] | Methoxetamine | 248 | 76 | 12 | Qualifier | 121 | 39 | 8 | Ketamine-D4 | 242/129 | 300 |
| | | | | | Qualifier | 175 | 25 | 12 | | | |
| | | | | | Quantifier | 179 | 22 | 9 | | | |
| 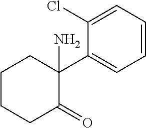 [SMILES: NC1(c2ccccc2Cl)CCCCC1=O] | Norketamine | 224 | 70 | 10 | | | | | Ketamine-D4 | 242/129 | 300 |
| | | | | | Qualifier | 125 | 30 | 12 | | | |
| | | | | | Quantifier | 173 | 19 | 12 | | | |
| 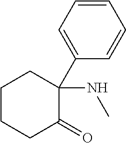 | Deschloroketamine | 204 | 65 | 10 | Qualifier | 145 | 23 | 10 | Ketamine-D4 | 242/129 | 300 |
| | | | | | Qualifier | 91 | 35 | 7 | | | |
| | | | | | Quantifier | 145 | 18 | 10 | | | |
| 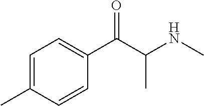 | Mephedrone | 178 | 80 | 10 | Qualifier | 160 | 28 | 10 | Ketamine-D4 | 242/129 | 300 |
| | | | | | Qualifier | 91 | 47 | 10 | | | |
| | | | | | Quantifier | 188 | 17 | 13 | | | |
| 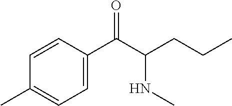 | 4-MPD | 206 | 79 | 8 | Qualifier | 145 | 30 | 10 | Ketamine-D4 | 242/129 | 300 |
| | | | | | Qualifier | 105 | 28 | 7 | | | |
| | | | | | Quantifier | 202 | 19 | 14 | | | |
| 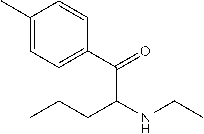 [SMILES: CCCC(NCC)C(=O)c1ccc(C)cc1] | MEAP | 220 | 80 | 12 | Qualifier | 105 | 31 | 7 | Ketamine-D4 | 242/129 | 300 |
| | | | | | Qualifier | 160 | 25 | 11 | | | |
| | | | | | Qualifier | 175 | 20 | 12 | | | |
| | | | | | Quantifier | 180 | 16 | 12 | | | |
| 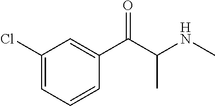 | CMC | 198 | 80 | 9 | Qualifier | 145 | 26 | 10 | Ketamine-D4 | 242/129 | 300 |
| | | | | | Qualifier | 144 | 43 | 10 | | | |
| | | | | | Quantifier | 132 | 37 | 10 | | | |
| 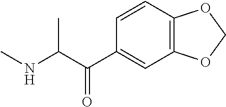 | Methylone | 208 | 80 | 10 | Qualifier | 160 | 24 | 10 | Ketamine-D4 | 242/129 | 300 |
| | | | | | Qualifier | 190 | 17 | 10 | | | |
| | | | | | Quantifier | 202 | 25 | 14 | | | |
| 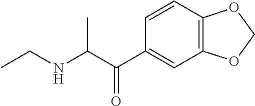 [SMILES: CCNC(C)C(=O)c1ccc2c(c1)OCO2] | Ephylone | 250 | 72 | 11 | Qualifier | 232 | 20 | 9 | Ketamine-D4 | 242/129 | 300 |
| | | | | | Qualifier | 189 | 33 | 13 | | | |
| | | | | | Quantifier | 160 | 35 | 11 | | | |
| 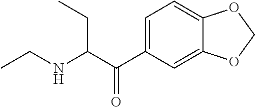 [SMILES: CCNC(CC)C(=O)c1ccc2c(c1)OCO2] | Eutylone | 236 | 70 | 9 | Qualifier | 218 | 20 | 10 | Ketamine-D4 | 242/129 | 300 |
| | | | | | Qualifier | 188 | 25 | 13 | | | |
| | | | | | Quantifier | 140 | 36 | 10 | | | |
| 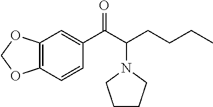 [SMILES: CCCCC(C(=O)c1ccc2c(c1)OCO2)N1CCCC1] | 3,4-MDP HP | 290 | 102 | 7 | Qualifier | 219 | 25 | 15 | Ketamine-D4 | 242/129 | 300 |
| | | | | | Qualifier | 189 | 30 | 14 | | | |
| | | | | | Qualifier | 135 | 35 | 10 | | | |
| 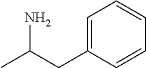 | Amphetamine | 136 | 50 | 10 | Quantifier | 91 | 21 | 10 | Ketamine-D4 | 242/129 | 300 |
| | | | | | Qualifier | 119 | 11 | 10 | | | |
| 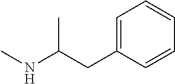 | Methamphetamine | 150 | 80 | 10 | Quantifier | 119 | 13 | 10 | Methamphetamine-D8 | 158/93 | 300 |
| | | | | | Qualifier | 91 | 30 | 10 | | | |
| 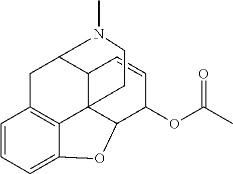 [SMILES: CC(=O)OC1C=CC2C3Cc4cccc5c4C2(CCN3C)C1O5] | 6-acetylmorphine | 328 | 138 | 9 | Quantifier | 211 | 35 | 15 | Methamphetamine-D8 | 158/93 | 400 |
| | | | | | Qualifier | 193 | 37 | 14 | | | |
| | | | | | Qualifier | 165 | 51 | 11 | | | |
| 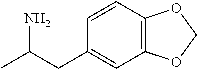 | MDA | 180 | 55 | 10 | Quantifier | 133 | 24 | 9 | Methamphetamine-D8 | 158/93 | 400 |
| | | | | | Qualifier | 135 | 25 | 10 | | | |
| | | | | | Quantifier | 163 | 16 | 12 | | | |
| 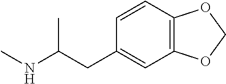 | MDMA | 194 | 65 | 12 | Qualifier | 133 | 27 | 10 | Ketamine-D4 | 242/129 | 300 |
| | | | | | Qualifier | 135 | 28 | 10 | | | |
| | | | | | Quantifier | 91 | 27 | 6 | | | |
| 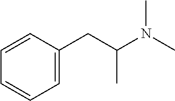 | DMA | 164 | 70 | 11 | Qualifier | 119 | 17 | 8 | Ketamine-D4 | 242/129 | 300 |
| | | | | | Qualifier | 65 | 56 | 8 | | | |
| | | | | | Quantifier | 121 | 30 | 9 | | | |
| 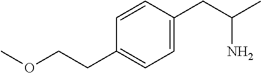 | PMEA | 194 | 66 | 9 | Qualifier | 149 | 17 | 11 | Ketamine-D4 | 242/129 | 300 |
| | | | | | Qualifier | 91 | 46 | 10 | | | |
| 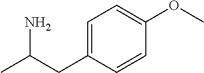 | PMA | 166 | 53 | 7 | Quantifier | 121 | 26 | 10 | Ketamine-D4 | 242/129 | 300 |
| | | | | | Qualifier | 91 | 40 | 8 | | | |
| | | | | | Qualifier | 78 | 53 | 10 | | | |
| 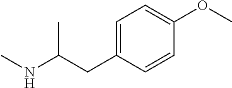 | PMMA | 180 | 56 | 13 | Quantifier | 149 | 16 | 10 | Ketamine-D4 | 242/129 | 300 |
| | | | | | Qualifier | 91 | 42 | 9 | | | |
| | | | | | Qualifier | 78 | 53 | 10 | | | |
| 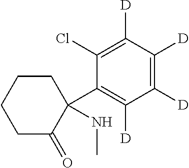 [SMILES: [2H]c1c([2H])c([2H])c(C2(NC)CCCCC2=O)c(Cl)c1[2H]] | Ketamine-D4 | 242 | 80 | 10 | | 129 | 38 | 10 | | | 300 |
| | | | | | | | | | Internal standards | | |
| 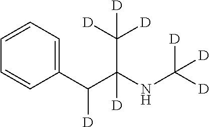 [SMILES: [2H]C(c1ccccc1)C([2H])(NC([2H])([2H])[2H])C([2H])([2H])[2H]] | Methamphetamine-D8 | 158 | 62 | 8 | | 93 | 18 | 7 | | | 400 |
| | | | | | | 124 | 35 | 9 | | | |

*Quantifier for quantity; Qualifier for quality.

### Validation of Forensic Toxicological Methods

The validation studies design and follow the standard practices and recommendations from the Scientific Working Group for Forensic Toxicology (SWGTOX) Standard Practices for Method Validation in Forensic Toxicology. Blank matrices, pooled by healthy volunteers' urine collection, were used in all validation parameters, including linearity, accuracy, precision, etc.

Multiple-point calibration standards for the 21 analytes were prepared over a concentration range from 1 to 1000 ng/mL (1, 5, 10, 20, 50, 75, 100, 200, 400, 500, 750, 1000 ng/mL). Each calibration point was run in quintuplicate. The normalized area for each analyte was calculated using the selected internal standard signal area (Supplementary Table 1). The plots were generated by the normalized area of targeted fragments against nominal spiked concentration. Linear calibration curves were constructed using a least square regression model with the reciprocal of the spiked concentration (1/x) as a weighting factor. The calibration parameter coefficient of determination (R²) was used to roughly estimate the linearity.

The limit of detection (LOD) and was estimated using the ratio of the average normalized area with 3.3 times the standard deviation of blanks within five measurements. The limit of quantification (LLOQ) was defined as the standard's concentration at which produced a bias within ±20%. For all the analytes, interference, carryover effect, accuracy, system precision, repeatability, matrix effect, and inter-day precision were evaluated at several different concentrations, including LLOQ.

The interference was evaluated by blank urine samples from three males and four females at different ionization temperatures. Accuracy, expressed as bias %, compared the concentration calculated from the linear model and the nominal concentration. Precision showed as the coefficient of variation (% CV) was assessed the level of variation at each concentration. System precision was used to assess the stability of the pDART system within five measurements by a single testing sample. Method precision revealed the variation within three independent preparations. Matrix effect, or ion suppression, was investigated by comparing the 50% methanol spiked with the analytes with blank spiked ones. Inter-day precision was used to evaluate the stability of the prepared solution. The carryover effect was assessed by measurement of the double blank solution (pooled urine without any intentional addition of analytes and internal standard) was conducted in the same testing set after the highest concentration samples (1000 ng/mL) used in calibration curves. Stability is investigated through the inter-day precision and method bias analyzed same test solution storage at −20° C. for 3 days in quintuplicate. Quality controls analyzed in triplicate at the concentration, 20, 40, 75, 200, 400, 750 ng/mL, were used to confirm the deflection of the linearity against the default linearity within the range of 0.8-1.2.

### Example 1

### Method Development

Preliminary experiments were carried out to find the best instrumental conditions for the detection of NPSs. With its low proton affinity, all NPSs were analyzed by operating in the positive mode. The method was optimized in terms of ionization helium temperature, DP, CE, and scan speed. The DP and CE values were optimized via the ESI ionization source and confirmed the fragmentation by the DART resource. Gas temperature was optimized to be as low as possible while still producing a stable signal. Lower voltages were also desirable to decrease the interference of the unexpected heat degradation. The ionization temperature was adjusted empirically, 300° C. and 400° C. were found to acquire adequate intensity in different NPS.

Notably, the preliminary experiments showed a strong background signal in the blank, which is a urine mixture pooled by multiple healthy human samples to simulated the real testing scenario. To alleviate the background noise, the characteristics of papers as the loading materials have been investigated.

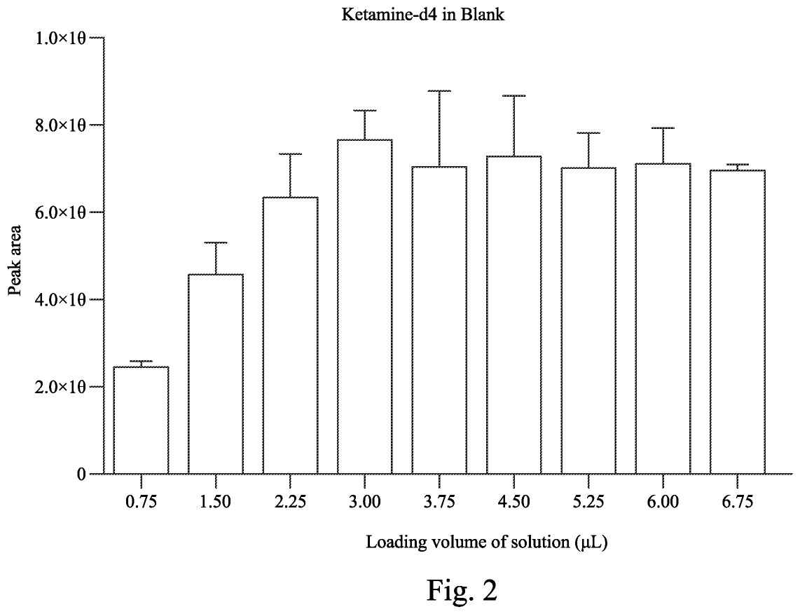

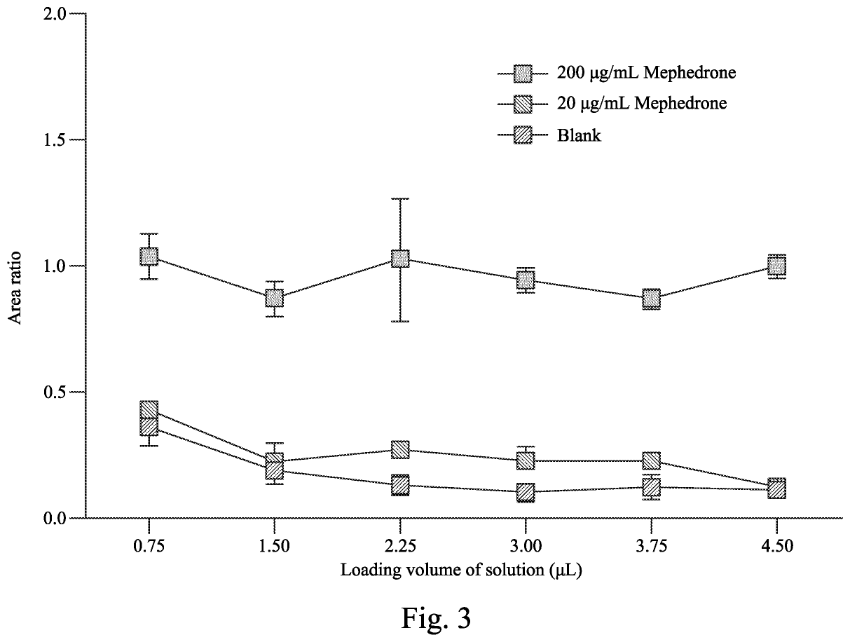

Biological sample was loading on the chromatography paper with multiple drying and sampling process to enhance the overall amount of loading volume where the analyte was captured and preconcentrated while the matrix was absorbed into the paper. To increase the sensitivity of the method, the number of times of sampling was assessed through sampling the fixed matrix volume each time and monitoring the frequency of repetition. The overall sampling volume against the area under curve (AUC) of Ketamine-d4 transition was plotted in FIG. 2. The intensity has been maximized at a total 3.0 μL solution volume. The signal intensity of the Mephedrone (m/z 178→160, that is, first m/z is 178, second m/z is 160) in a low concentration (20 μg/mL) can discriminate with the signal of the urine blank (FIG. 3). The sensitivity of the methodology was improved by increasing the overall sampling volume on the paper.

### Example 2

### Analytical Performance and Validation

Prior to application in forensic casework, analytic methods require reliability, robustness, and accuracy of analytical validation data to test the robustness and suitability of the strategy. To appraise the performance of real-time screening under real-world circumstances, validation was established with the spiked-in pooled urine blank. The quantitative approach required the addition of the internal standard mixture directly to the sample collector before sampling. The protocol was delineated by the recommendation from Standard Practices for Method Validation in Forensic Toxicology (SWGTOX).

### Linearity and Selectivity/Interference

Selectivity was an important aspect of the analytic method, and thorough validation of this parameter was required to prevent false-positive findings, especially when no separation methods are applied prior to MS analysis. To assess method specificity towards endogenous compounds, urine sample/double blank from 4 healthy females and 3 healthy males were analyzed. No unexpected interference was observed in all analytes due to the MRM transitions were used in each analyte, demonstrating a good selectivity of the method. To be notable, some interference in the double blank (not containing internal standard nor analyte) in the internal standard Ketamine-d4 at gas temperature 400° C.; therefore, the internal standard replaced to Methamphetamine-d8 form Ketamine-d4 in the analytes tested at 400° C. and 300° C., respectively.

Linearity is constructed to evaluate the suitability of the quantitative approach. The testing range for linearity spanned 3 orders of magnitude. The estimated linear parameters and the R² values were obtained from the calibration curves by normalized area of analyte versus respectively internal standard versus nominal concentrations and fitted by least-squares linear regression with weighting factor 1/x. Good linear fits were obtained for all the analytes under this scenario in the R² values ranged from 0.8343 (Amphetamine) to 0.9963 (PMMA) (Table 2).

TABLE 2
Validation results, linear parameters, LOD, and other validation parameters at LLOQ, 400 ng/mL.

| | Linearity | | | | | LLOQ | | | | |
|---|---|---|---|---|---|---|---|---|---|---|
| | | | | | calcd. | | System | Method | | Matrix |
| | Range | | | | LOD | Conc. | precision | precision | Accuracy | Effect |
| Compound | (ng/mL) | Slope | Intercept | R square | (ng/mL) | (ng/mL) | (% CV) | (% CV) | (% Bias) | (%) |
| Ketamine | 5-1000 | 0.0089 | 0.0046 | 0.9916 | 2 | 20 | 2.65 | 6.31 | 1.72 | 116.75 |
| Methoxetamine | 1-1000 | 0.0153 | 0.0022 | 0.9912 | 3.06 | 20 | 4.62 | 7.85 | −1.4 | 106.05 |
| Norketamine | 1-1000 | 0.0069 | 0.0018 | 0.9938 | 3.92 | 50 | 5.69 | 12.76 | −3.86 | 139.32 |
| Deschloroketamine | 1-1000 | 0.0078 | 0.0011 | 0.9916 | 2.44 | 50 | 3.77 | 13.07 | −3.17 | 87.53 |
| Mephedrone | 10-1000 | 0.0034 | 0.0017 | 0.9867 | 6.43 | 20 | 6.87 | 16.4 | −1.87 | 42.44 |
| 4-MPD | 10-1000 | 0.0597 | 0.0756 | 0.9874 | 8.91 | 20 | 12.56 | 16.88 | 12.16 | 105.05 |
| MEAP | 5-1000 | 0.0764 | 0.0407 | 0.9923 | 3.97 | 20 | 5.21 | 9.06 | 0.58 | 103.45 |
| CMC | 10-1000 | 0.0047 | −0.0027 | 0.9929 | 9.52 | 20 | 7.86 | 18.98 | −4.2 | 43.13 |
| Methylone | 10-1000 | 0.0015 | 0.0028 | 0.9828 | 28.75 | 50 | 5.35 | 10.54 | −0.69 | 49.45 |
| Ephylone | 1-1000 | 0.0146 | 0.0003 | 0.9913 | 0.38 | 20 | 6.64 | 16.41 | −3.32 | 63.77 |
| Eutylone | 10-1000 | 0.0015 | 0.0018 | 0.9862 | 11.81 | 20 | 5.44 | 16.94 | 7.13 | 43.86 |
| 3,4-MDPHP | 1-1000 | 0.0066 | −0.0003 | 0.983 | 0.42 | 20 | 4.14 | 13.15 | −4.5 | 96.76 |
| Amphetamine | 10-1000 | 0.0074 | 0.0884 | 0.8343 | 113.33 | 75 | 10.14 | 18.36 | 54.38 | 143.61 |
| Methamphetamine | 5-1000 | 0.0037 | 0.001 | 0.9909 | 5.94 | 20 | 3.08 | 14.39 | −0.65 | 122.2 |
| 6-acetylmorphine | 10-1000 | 0.0002 | 0.0016 | 0.9321 | 66.39 | 50 | 17.8 | 28.39 | 19.62 | 136.49 |
| MDA | 10-1000 | 0.0009 | 0.003 | 0.9822 | 21.46 | 50 | 10.51 | 21.99 | 0.15 | 102.18 |
| MDMA | 5-1000 | 0.0021 | 0 | 0.9958 | 3.21 | 20 | 7.79 | 15.26 | 4.23 | 52.17 |
| DMA | 10-1000 | 0.0124 | 0.0285 | 0.9836 | 25.26 | 50 | 4.07 | 14.12 | 4.19 | 101.47 |
| PMEA | 10-1000 | 0.0072 | 0.0259 | 0.9781 | 25.84 | 50 | 9.64 | 12.04 | 14.66 | 126.83 |
| PMA | 10-1000 | 0.0036 | 0.0109 | 0.9822 | 38.83 | 50 | 14.08 | 17.56 | 8.76 | 136.59 |
| PMMA | 1-1000 | 0.0063 | 0.0007 | 0.9963 | 2.41 | 20 | 3.76 | 14.44 | 0.15 | 99.57 |

| | LLOQ | | 400 ng/mL | | | | | |
|---|---|---|---|---|---|---|---|---|
| | Inter-day | | System | Method | | Matrix | Inter-day | |
| | precision | Stability | precision | precision | Accuracy | Effect | precision | Stability |
| Compound | (% CV) | (% Bias) | (% CV) | (% CV) | (% Bias) | (%) | (% CV) | (% Bias) |
| Ketamine | 10.96 | 11.63 | 2.77 | 15.71 | 1.35 | 105.47 | 18.27 | 4.64 |
| Methoxetamine | 8.63 | 7.38 | 3.18 | 15.28 | 0.75 | 115.76 | 21.14 | 20.36 |
| Norketamine | 10.44 | 3.96 | 4.83 | 12.44 | −0.3 | 145.55 | 17.43 | 13.81 |
| Deschloroketamine | 10.81 | −6.03 | 9.56 | −3.34 | 20.17 | 86.69 | 11.92 | 6.21 |
| Mephedrone | 12.39 | 6.12 | 5.15 | 10.42 | −0.01 | 39.26 | 12.29 | −1.57 |
| 4-MPD | 5.52 | −7.57 | 13.41 | 16.98 | −5.89 | 73.6 | 19.84 | −5.94 |
| MEAP | 8.58 | −4.72 | 9.35 | 12.81 | 2.76 | 77.33 | 18.02 | −8.65 |
| CMC | 16.77 | 10.13 | 7.03 | 13.68 | −2.78 | 44.96 | 18.8 | 15.98 |
| Methylone | 17.01 | 10.41 | 4.56 | 10.66 | 1.99 | 35.27 | 12.97 | 8.81 |
| Ephylone | 9.31 | 1.62 | 10.05 | 18.05 | −8.55 | 54.03 | 18.69 | −8.94 |
| Eutylone | 13.81 | −2.43 | 10.24 | 18.56 | 5.96 | 42.62 | 20.84 | −5.46 |
| 3,4-MDPHP | 11.38 | 0.06 | 10.81 | 26.07 | −6.52 | 64.47 | 22.86 | 13.16 |
| Amphetamine | 25.7 | −17.07 | 5.41 | 10.91 | 2.39 | 121.03 | 12.89 | 11.74 |
| Methamphetamine | 15.23 | 9.79 | 8.02 | 11.34 | 3.39 | 107.8 | 13.19 | −0.85 |
| 6-acetylmorphine | 23.96 | 2.83 | 20.83 | 25.07 | 0.36 | 109.42 | 23.61 | −2.73 |
| MDA | 10.75 | 2.76 | 8.46 | 16.43 | 5.27 | 114.31 | 18.63 | 1.12 |
| MDMA | 11.53 | 12.94 | 8.29 | 8.85 | −0.48 | 53.3 | 17.18 | −8.26 |
| DMA | 6.66 | −0.94 | 8.06 | 12.79 | −4.08 | 94.46 | 8.14 | 14.17 |
| PMEA | 14.54 | 4.9 | 7.93 | 9.19 | −3.9 | 95.33 | 9.82 | 12.77 |
| PMA | 15.52 | 14.85 | 2.98 | 13.42 | −6.19 | 97.28 | 13.85 | −17.6 |
| PMMA | 16.01 | −1.9 | 5.47 | 12.87 | 1.45 | 90.2 | 12.41 | 21.71 |

Sensitivity was assessed by limits of detection and limits of quantitation. Limit of detection (LOD) was established using the statistical analysis of background. The average and standard deviation of the signal of pooled urine matrix is analyzed in five measurements. Below 40 ng/mL, the calculated LODs of most targeted drugs achieved the sensitivity for a toxicological application. Amphetamine and 6-acetylmorphine reported at the level of 113.33 ng/mL and 66.39 ng/mL, respectively, also achieved the sensitivity for a toxicological application. This result of amphetamine was attributed to strong interference from the chromatography paper. Solutions of decreasing concentration (low to 20 ng/mL) were analyzed. For most of the compounds, the lowest concentration of standard at which the bias within ±15% was determined as the LLOQ.

### Accuracy, Precision and Other Features

Accuracy, precision, and other validation features were tested at different concentrations including LLOQ, 400 ng/mL, and 750 ng/mL for each analyte. All the detailed results of the validation are reported in Table 2. No carryover effect was observed for the experiment after treating the upper concentration 1000 ng/mL used to make the calibration curves. The result was consistent with the facture of DART ionization in a noncontact fashion with minimal memory effects. Regarding accuracy, biases were within the ±15% at each level. Amphetamine produced a bias at 54.38% at 75 ng/mL, and the bias was within ±15% at the medium concentration (400 ng/mL). Precision was divided into instrument precision (or system precision), method precision, and inter-day precision. The methodology shows good precision. For all species ionized at 300° C., the % CV values of the quantity method precision were lower than 20% at the LLOQ level. 6-acetylmorphine and MDA analyzed at 400° C. were beyond 20% CV values of the quantitative concentration method precision, that were also beyond the criterion level. The results and the high calculated LOD of 6-acetylmorphine might be caused by the potential for chemical interference at a higher ionization temperature. The test solutions were considered stable via the results of inter-day % CV value falling within ±30% and % bias value limited in ±30% at each concentration. Concerning the matrix effect, intense results are expected due to a lack of separation or sample purification. Most compounds showed a negative matrix effect meaning that ion suppression occurs when samples were prepared in urine compared to 50% methanol. By contrast, four analytes, Norketamine, Amphetamine, 6-acetylmorphine, PMA, presented positive matrix effects near to 140% at the LLOQ level.

### Example 3

### Evaluation on Real Samples

We analyzed 40 urine collected form drug abused subjects with validated method. Internal standards were added to matrixes before sampling. The prediction was evaluated by using all the transitions of each analyte.

The qualitative results of the pDART with LRMS drug screen and the comparison with LC-MS/MS were presented in Table 3. The results of urine specimens were compared with well-studied and validated LC-MS/MS analysis described above. Of pDART screening detections, compounds not detected by the LC-MS/MS conformity test were treated as false positive (FPs). Of LC-MS/MS confirmation, drugs not detected by pDART screening were considered as false negatives. The results of the pDART screen and LC-MS/MS method were reported in the 40 samples, and the difference in sensitivity between the two methods was presented. LC-MS/MS exhibited high sensitivity for low concentrations of drugs in urine; in contrast, the sensitivity of the pDART screening method is in the range of 10-40 ng/mL. For instance, of the screening result of Deschloroketamine, 3 false negatives detected by the LC-MS/MS assay were at concentrations below the pDART detection limit.

TABLE 3
Qualitative comparison of the pDART screen with LC-MS/MS in 40 urine specimens.

| | Predict | | L C-MS/MS | | | | | | |
|---|---|---|---|---|---|---|---|---|---|
| Compound | Positive | Negative | TP | FP | TN | FN | PPA | NPA | Accuracy |
| Ketamine | 16 | 24 | 14 | 2 | 22 | 2 | 87.50% | 91.67% | 90.00% |
| Methoxetamine | 0 | 40 | 0 | 0 | 40 | 0 | NA | 100.00% | 100.00% |
| Norketamine | 21 | 19 | 17 | 4 | 18 | 1 | 94.44% | 81.82% | 87.50% |
| Deschloroketamine | 1 | 39 | 1 | 0 | 36 | 3 | 25.00% | 100.00% | 92.50% |
| Mephedrone | 18 | 22 | 13 | 5 | 21 | 1 | 92.86% | 80.77% | 85.00% |
| 4-MPD | 0 | 40 | 0 | 0 | 40 | 0 | NA | 100.00% | 100.00% |
| MEAP | 2 | 38 | 2 | 0 | 37 | 1 | 66.67% | 100.00% | 97.50% |
| CMC | 0 | 40 | 0 | 0 | 40 | 0 | NA | 100.00% | 100.00% |
| Methylone | 13 | 27 | 2 | 11 | 27 | 0 | 100.00% | 71.05% | 72.50% |
| Ephylone | 2 | 38 | 1 | 1 | 37 | 1 | 50.00% | 97.37% | 95.00% |
| Eutylone | 14 | 26 | 14 | 0 | 25 | 1 | 93.33% | 100.00% | 97.50% |
| 3,4-MDPHP | 0 | 40 | 0 | 0 | 40 | 0 | NA | 100.00% | 100.00% |
| Amphetamine | 9 | 31 | 8 | 1 | 28 | 3 | 72.73% | 96.55% | 90.00% |
| Methamphetamine | 16 | 24 | 12 | 4 | 19 | 5 | 70.59% | 82.61% | 77.50% |
| 6-acetylmorphine | 0 | 40 | 0 | 0 | 39 | 1 | 0.00% | 100.00% | 97.50% |
| MDA | 4 | 36 | 0 | 4 | 36 | 0 | NA | 90.00% | 90.00% |
| MDMA | 2 | 38 | 0 | 2 | 37 | 1 | 0.00% | 94.87% | 92.50% |
| DMA | 16 | 24 | 5 | 11 | 23 | 1 | 83.33% | 67.65% | 70.00% |
| PMEA | 0 | 40 | 0 | 0 | 40 | 0 | NA | 100.00% | 100.00% |
| PMA | 2 | 38 | 2 | 0 | 38 | 0 | 100.00% | 100.00% | 100.00% |
| PMMA | 10 | 30 | 1 | 9 | 30 | 0 | 100.00% | 76.92% | 77.50% |

TP: true positive; FP: false positive; TN: true negative; FN: false negative; PPA: PPA: positive percent agreement; NPA: negative percent agreement.
*CI: confidence interval

Qualitatively, the rates of agreement for positive and negative results can be used in measuring a method's positive percent agreement, negative percent agreement and overall percent agreement. The positive percent agreement of Methoxetamine, 4-MPD, CMC, 3,4-MDPHP, PMEA could not be obtained due to the lack of positive specimen; The negative percent agreement was 100%. The overall percent agreement of the pDART rapid screen method ranged from 70.0 to 100.0% for all evaluated drugs.

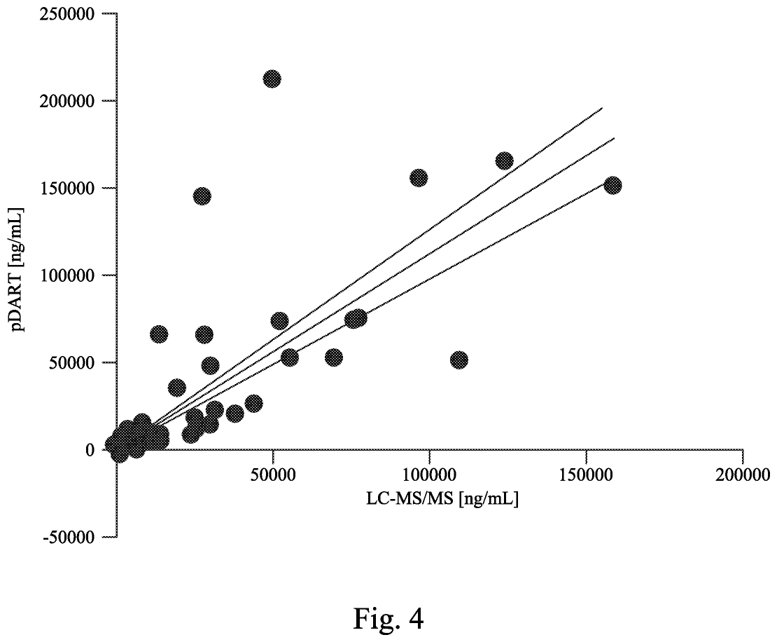

The quantitative results obtained by the pDART screening method were compared to the LC-MS/MS confirmations results using weighted least squares regression. The correlations of the two methods were assessed with a Pearson's correlation coefficient. The pDART method was developed as a rapid screening method whereas the HPLC method was developed for quantitative confirmation. The overall weighted Kappa coefficient reached 0.8152 through separating to 6 concentration levels (FIG. 4).

### Example 4

### Screening Non-Targeted NPSs Through Precursor Ion Scanning Approach

The precursor ion (PI) scanning mode is valuable for investigating groups of compounds that generate common product ions after fragmentation in complex matrices. The approach was based on an automatic structural analysis using the hybrid triple quadrupole linear ion trap technology of the QTRAP system. 24 The information-dependent acquisition-enhanced product ion (IDA-EPI) scan was performed using a combination of precursor ion screening experiments of the daughter ions of interest with the acquisition of the daughter ion spectrum of these selective parent ions in urine. The product ions were selected via the research of fragmentation pathway on emerging synthetic cathinone derivatives.

Product ion of m/z 135 was chosen for methylenedioxy moiety. The methylenedioxy-containing synthetic cathinones, dibutylone and MDPV, were detected under the paper-loaded screening method without pre-separation of chromatography. Pyrrolidine ring-containing synthetic cathinones, alpha-PVP, 4-chloro-alpha-PVP were detected under PI scan for pyrrolidine moiety (m/z 70); however, MDPV cannot be detected under scanning for m/z 70 but m/z 125 (n-butylidenepyrrolidinium moiety) due to the messy result on low mass scanning in PI mode. The detailed experiments parameters and spectra can be found in Table 4.

TABLE 4
Comparison of quantitative results of pDART screen and
LC-MS/MS confirmation

| | | Correlation | | | |
|---|---|---|---|---|---|
| Compound | Number | coefficient | Equation | Slope (95% CI) | Intercept (95% CI) |
| Ketamine | 16 | 0.937 | Y = 0.5497*X − 129.5 | 0.4327 to 0.6667 | −602.7 to 343.6 |
| Norketamine | 18 | 0.903 | Y = 0.4610*X + 144.7 | 0.3449 to 0.5770 | −187.2 to 476.7 |
| Deschloroketamine | 4 | 1.000 | Y = 0.3166*X + 2.991 | 0.2912 to 0.3421 | −0.7427 to 6.725 |
| Mephedrone | 14 | 0.712 | Y = 1.705*X + 9191 | 0.6483 to 2.761 | −31088 to 49470 |
| MEAP | 3 | 1.000 | Y = 0.6577*X − 156.9 | 0.5374 to 0.7781 | −2277 to 1964 |
| Eutylone | 15 | 0.949 | Y = 1.029*X + 4069 | 0.8235 to 1.235 | −8954 to 17091 |
| Amphetamine | 11 | 0.976 | Y = 0.5643*X + 216.5 | 0.4691 to 0.6595 | −715.4 to 1148 |
| Methamphetamine | 19 | 0.985 | Y = 0.4835*X + 541.1 | 0.4399 to 0.5271 | −705.6 to 1788 |
| DMA | 6 | 0.952 | Y = 1.688*X − 71.91 | 0.9336 to 2.443 | −510.3 to 366.5 |
| Overall | 59 | 0.807 | Y = 1.663*X + 731.0 | 1.253 to 2.073 | −6676 to 8138 |

*CI: confidence interval

The present disclosure provides an economical, easy-to-operate method and platform for screening new psychoactive substance. Compared with traditional chromatography analysis technology, the present disclosure has greatly shortened the sample analysis time. It only takes 30 seconds to complete a sample analysis, and time cost is successfully reduced. The operation of platform and method the present disclosure is simple, the platform is stable, and the minimum detection concentration is low.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of screening new psychoactive substance, comprising:
providing a sample;
placing the sample on a chromatographic paper;
ionizing the sample on the chromatographic paper by a direct analysis in real time (DART);
performing a mass spectrometry analysis on the ionized sample to obtain a sample mass spectrum; and
comparing a known standard mass spectrum with the sample mass spectrum, wherein when a profile of the known standard mass spectrum is the same as a profile of the sample mass spectrum, and the known standard mass spectrum is not exactly the same as the sample mass spectrum, the sample is determined to be the new psychoactive substance.

2. The method of claim 1, wherein an ionization temperature of the direct analysis in real time is from 300° C. to 400° C.

3. The method of claim 1, wherein the direct analysis in real time comprises ionizing by a positive ionization mode.

4. The method of claim 3, wherein the direct analysis in real time comprises ionizing by a multiple reaction monitoring (MRM) mode in the positive ionization mode.

5. The method of claim 1, wherein a known standard of the known standard mass spectrum comprises a known drug.

6. The method of claim 5, wherein the known drug comprises ketamine, methoxetamine, norketamine, deschloroketamine, mephedrone, 4-methylpentedrone (4-MPD), 4-methyl-α-ethylaminopentiophenone (MEAP), chloromethcathinone (CMC), 3,4-methylenedioxy-N-methylcathinone (methylone), ephylone, eutylone, 3,4-Methylenedioxy-α-pyrrolidinohexiophenone (3,4-MDPHP), amphetamine, methamphetamine, 6-acetylmorphine, 3,4-methylenedioxyamphetamine (MDA), 3,4-methylenedioxymethamphetamine (MDMA), para-methoxyethylamphetamine (PMEA), para-methoxyamphetamine (PMA), para-methoxymethamphetamine (PMMA), or a combination thereof.

7. The method of claim 1, wherein the mass spectrometry analysis is an ion trap mass spectrometry analysis.

8. The method of claim 1, wherein the profile of the known standard mass spectrum comprises one or more than one mass to charge ratio (m/z).

9. A platform of screening new psychoactive substance, comprising:
a carrier loading at least one chromatographic paper;
a DART device adjacent to the carrier;
a mass spectrometer adjacent to the DART device, wherein a sample placed on the chromatography paper and ionized by the DART device is analyzed by the mass spectrometer to obtain a sample mass spectrum; and
a drug screening model comprising a computer processor and a memory, the memory storing a plurality of computer program instructions that, when executed by the computer processor, cause the computer processor to implement following steps, comprising:
importing the sample mass spectrum; and
comparing a known standard mass spectrum with the sample mass spectrum, wherein when a profile of the known standard mass spectrum is the same as a profile of the sample mass spectrum, and the known standard mass spectrum is not exactly the same as the sample mass spectrum, the sample is determined to be the new psychoactive substance.

10. The platform of claim 9, wherein the at least one chromatographic paper is a plurality of chromatographic papers, wherein the carrier comprises a body and a plurality of paper modules sequentially disposed on the body, wherein the plurality of chromatography papers are respectively disposed on the plurality of paper modules.

11. The platform of claim 9, wherein the mass spectrometer comprises a high-resolution mass spectrometer or a low-resolution mass spectrometer, wherein
the high-resolution mass spectrometer provides a mass accuracy level below 5 ppm and a mass resolution above 10,000 m/Δm;
the low-resolution mass spectrometer provides a mass accuracy level above or equal to 5 ppm and a mass resolution below or equal to 10,000 m/Δm.

12. The platform of claim 11, wherein the low-resolution mass spectrometer comprises a triple quadrupole linear ion trap mass spectrometer.

13. The platform of claim 9, wherein the profile of the known standard mass spectrum comprises one or more than one mass to charge ratio (m/z).

14. The platform of claim 9, wherein a known standard of the known standard mass spectrum comprises a known drug.

15. The platform of claim 14, wherein the known drug comprises ketamine, methoxetamine, norketamine, deschloroketamine, mephedrone, 4-MPD, MEAP, CMC, methylone, ephylone, eutylone, 3,4-MDPHP, amphetamine, methamphetamine, 6-acetylmorphine, MDA, MDMA, PMEA, PMA, PMMA, or a combination thereof.
